(19) United States
(12) Patent Application Publication
(10) Pub. No.: US 2004/0036038 A1
(43) Pub. Date: Feb. 26, 2004

(54) Method and apparatus for plasma doping

(76) Inventors: Tomohiro Okumura, Kadoma-shi (JP); Ichiro Nakayama, Kadoma-shi (JP); Bunji Mizuno, Ikoma-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/611,867

(22) Filed: Jul. 3, 2003

(30) Foreign Application Priority Data
Jul. 11, 2002 (JP) 2002-202484

(51) Int. Cl. A61N 5/00; G21G 5/00; H01L 21/26; H01L 21/42; G01K 1/08; H01J 3/14

(52) U.S. Cl. 250/492.2; 438/513; 250/397

(57) ABSTRACT

According to a method for impurity implantation, a substrate is positioned on a table provided within a chamber in which a vacuum will be introduced and also an implantation impurity is supplied. A first high frequency electric power is applied to a plasma generating element to thereby cause a plasma so that the impurity in the chamber is implanted in the substrate. Also, a second high frequency electric power is applied to the table. Detected are a condition of the plasma in the chamber and a voltage or current in the table. Controller controls at least one of the first and second high frequency electric power according to the detected condition of the plasma and/or the detected voltage or current, thereby controlling an implantation concentration of the impurity to be implanted.

### RELATED APPLICATION

The present application claims the benefit of a patent application No. 2002-202484 filed in Japan on Jul. 11, 2002, the entire disclosure of which is incorporated herein by reference.

### FIELD OF THE INVENTION

The present invention relates to a method and apparatus for doping an impurity ion into a substrate such as semiconductor substrate by the use of a plasma doping, or plasma implantation technique.

### BACKGROUND OF THE INVENTION

U.S. Pat. No. 4,912,065 discloses a plasma doping by which an ionized impurity is implanted into a substrate with a reduced energy. Also, Japanese Patent No. 2,718,926 discloses a method for controlling a concentration of the implanted impurity, in which a high frequency current is measured while discharging and thereby it is controlled.

However, the control method has a disadvantage that changing the high frequency power and thereby controlling the high frequency current results in unwanted changes of electron density, impurity ion density in the plasma and ion energy to be applied to the substrate, causing an uncontrollability in the concentration.

### SUMMARY OF THE INVENTION

Therefore, an object of the present invention is to provide a method and apparatus in which a doping concentration can be controlled with ease.

According to a method and apparatus for plasma doping of the present invention, a substrate is positioned on a table provided within a chamber in which a vacuum will be introduced and also an implantation impurity will be supplied. A first high frequency electric power is applied to a plasma generating element to thereby cause a plasma in the chamber so that the impurity in the chamber is implanted in the substrate. Also, a second high frequency electric power is applied to the table. Detected are a condition of the plasma in the chamber and a voltage or current in the table. Controller controls at least one of the first and second high frequency electric power according to the detected condition of the plasma and/or the detected voltage or current, thereby controlling an implantation concentration of the impurity to be implanted.

In another aspect of the present invention, a voltage or current is detected in an electrode connected through a capacitor to the table. Then, the controller controls at least one of the first and second high frequency electric power according to the detected condition of the plasma and/or the detected voltage or current, thereby controlling an implantation concentration of the impurity to be implanted.

### BRIEF DESCRIPTION OF THE DRAWINGS

FIG. 1 is a schematic cross sectional view of a doping device according to the first embodiment of the present invention.

FIG. 2 is a graph showing an emission intensity versus boron concentration relationship.

FIG. 3 is a graph showing a high frequency voltage versus boron concentration relationship.

FIG. 4 is a schematic vertical cross sectional view of a doping device according to the second embodiment of the present invention.

FIG. 5 is a circuit diagram of a matching circuit and also shows a structure of a table.

FIG. 6 is a circuit diagram showing a modification of the matching circuit.

FIG. 7 is a schematic cross sectional view of a modification of the doping device.

### DETAILED DESCRIPTION OF THE PREFERRED EMBODIMENTS

With reference to the drawings, various embodiments of a method and apparatus for plasma doping of the present invention will be described hereinafter.

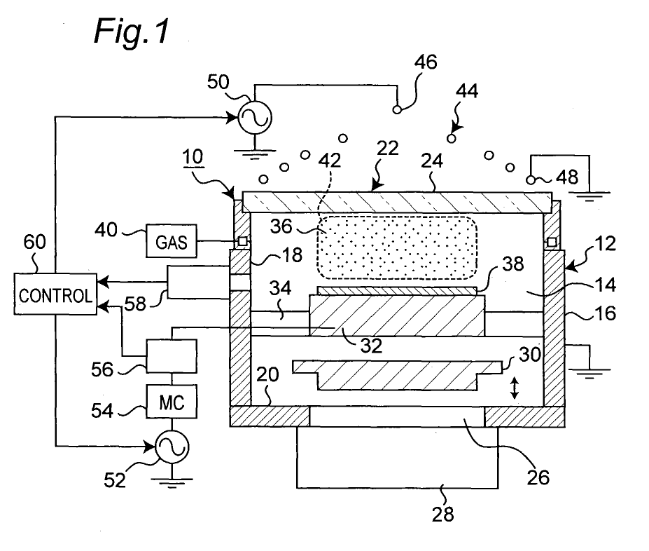

Referring to FIG. 1, there is shown a plasma doping device, generally indicated by reference numeral 10, according to the present invention. The doping device 10 has a cylindrical container 12 defining a chamber 14 therein. The container 12 has a first portion 16 defining side walls 18 and a bottom wall 20 of the container 12 and a second portion 22 defining a top wall 24 of the container 12. The first portion 16 of the container 12 is made of electrically conductive material such as aluminum and stainless steel and is electrically grounded to the earth. The second portion 22 of the container 12, i.e., top wall 24, is made of dielectric material such as silica glass, through which a high frequency electric field is induced in the chamber 14. The bottom wall 20 has an opening 26 defined therein and fluidly connected to a vacuum pump 28 such as turbo-molecular pump. Provided in the chamber 14 and adjacent to the opening 26 is a valve member 30 which is supported by an elevating device not shown so that an open ratio of the opening 26 and thereby the vacuum in the chamber 12 is controlled to a certain value such as 0.04 Pa by elevating the valve member 30.

Provided also in the chamber 14 is a table 32 which is made of electrically conductive material such as aluminum and stainless steel. The table 32 is supported at the center of the chamber 14 by a plurality of insulating supports 34 and spaced a certain distance away from the top dielectric wall 24 so that a certain volume of space 36 is defined for a plasma formation. Also, the table 32 has a top flat surface for supporting a substrate 38 such as silicon plate to which a predetermined ion is implanted.

A plasma gas supply source 40, i.e., impurity supply, is fluidly connected to the chamber 14 so that a certain gas including argon (Ar) and diborane (B₂H₆) is supplied therefrom into the chamber 14. For example, the amounts of argon and diborane gas are controlled to 10 sccm (standard cubic centimeters per minute) and 5 sccm, respectively.

In order to produce a plasma 42, in particular Inductively Coupled Plasma (ICP) in the plasma formation space 36, a plasma generating element or spiral coil 44 is arranged above the dielectric wall 24 and outside the chamber 14 in an coaxial fashion with the cylindrical container 12. As shown in the drawing, the central end portion 46 of the coil 44 is positioned higher than the opposite peripheral end portion 48 so that the coil 44 outlines a conical configuration. Also, the central end portion 46 of the coil 44 is connected to a first high frequency power source 50 capable of applying a high frequency electric power. Used for the first frequency power source 50 is a power source capable of controlling a voltage through a frequency control within in the frequency range of 300 kHz to 3 GHz or through a pulse width modulation in order to change a density of plasma generated in the chamber. In this embodiment, a frequency of 13.56 MHz is initially applied to the coil 44, for example. On the other hand, the peripheral end portion 48 of the coil 44 is grounded to the earth.

Also, in order to provide a negative polarity to the table 32 and the substrate 38 relative to the plasma 42 a second high frequency power source 52 is electrically connected to the table 32 through a matching circuit 54 and a voltage detector 56 (second monitor). The second high frequency power source 52, which is used for changing an ionization energy, may be a conventional power source which is similar to or different from that for the first high frequency power source. For example, a power source capable of controlling a voltage through a frequency control within in the frequency range of 300 kHz to 3 GHz or through a pulse width modulation is used. In this embodiment, a frequency of 600 kHz is initially applied to the table 32, for example. Also the implantation device 10 of this embodiment employs an optical emission spectroscopy for detecting a condition of plasma generated in the chamber 14 and then controlling a dose of ion implantation. To this end, a light detector 58 (first monitor) capable of detecting and measuring an amount of light emitted from the plasma in the chamber 14. The monitors 56 and 58 are connected to a controller 60, which in turn connected to the first and second power sources 50 and 52 for controlling the high frequency powers to be applied to the coil 44 and the table 32, respectively.

In operation of the ion implantation device 10 so constructed, the substrate 38 is positioned on the table 32 so that the substrate 38 makes a substantially full surface contact with opposing surface of the table 32. In this condition, the mixture of gas with Ar and B₂H₆ is supplied from the plasma gas supply source 40 into the chamber 14. Also, the chamber 14 is vacuumed by the pump 28 and the vacuum is controlled by the upward and/or downward movement of the valve member 30 and, as a result, by the adjustment of the opening ratio of the opening 26. Under the condition, once the high frequency power source 50 is turned on to induce the high frequency electric field in the chamber 14, the plasma 42 is generated above the substrate 38 in the space 36. Simultaneously generated between the plasma 42 and the substrate 38 is a sheath voltage, causing the boron implantation into the top surface of the substrate 38 to form an ultra thin boron implantation layer.

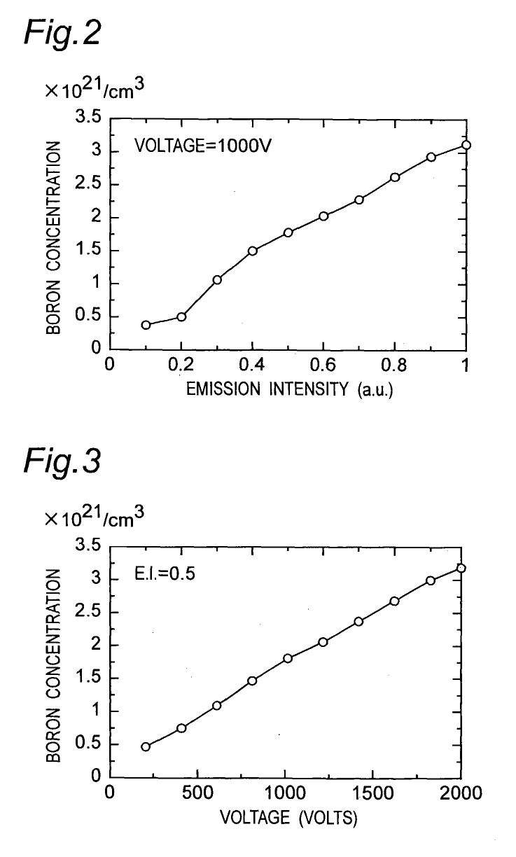

Using the implantation device, tests were made to determine a relationship between an emission intensity and an implanted boron concentration when 1,000 volts was applied to the table and substrate and a relationship between a voltage applied to the second high frequency power source and the Boron Concentration when the emission intensity of plasma was controlled at 0.5 (a.u.) by the control of AC power to the coil. The results are illustrated in FIGS. 2 and 3, respectively, which indicate that the boron concentration increased with the emission intensity and with the applied voltage. This means that each of the emission intensity and the voltage indicates a condition of the plasma and has a direct relationship with the boron concentration. This in turn means that the boron concentration is controlled by controlling the output of the first high frequency power source 50 corresponding to the light emission of the plasma 42 measured by the light detector 58, and/or by controlling the output of the second frequency power source 52, i.e., the voltage applied to the table 32 and measured by the voltage detector 56. Therefore, according to the implantation device 10 of the present invention, the controller 60 is programmed to control either or both of the outputs of the first and second high frequency power sources, 50 and 52, causing a desired dose of ion to be implanted in the surface of the substrate 38. Specifically, in this operation the first power source is feedback controlled to make the plasma vapor phase constant and also the second power source is feedback controlled to attain a constant voltage or power.

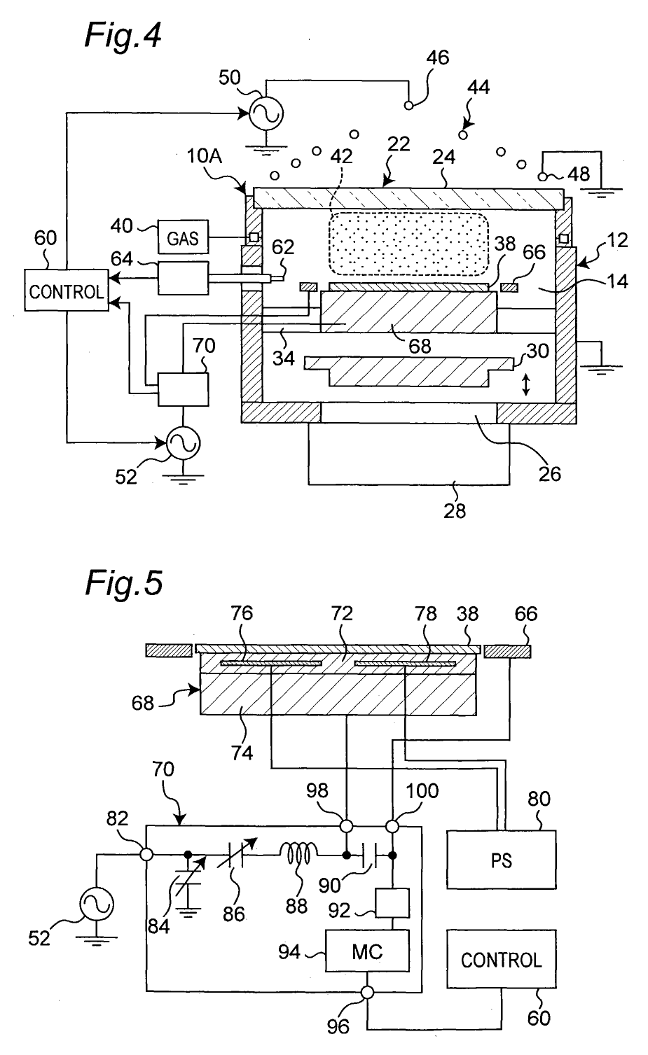

Referring to FIG. 4, another implantation device, generally indicated by reference numeral 10A, according to the second embodiment of the present invention will be described. In this embodiment, a single probe method is employed for detecting the condition of plasma generated in the chamber 14 and then controlling a dose of ion implantation. To this end, a single probe 62 with a rod-like electrode made of tungsten is projected in the chamber 14 and adjacent to the plasma formation space 36. Also, the probe 62 is electrically connected to a device 64 for monitoring a current density, which in turn connected to the controller 60. The current density corresponds to the emission intensity of the plasma, which means that the current density detected by the device 64 is used at the controller 60 for controlling the condition of the generated plasma and then the implanted boron concentration in the substrate.

In addition, an annular monitoring electrode 66 made of electrically conductive material is provided around a table 68 and also connected to a matching circuit 70. FIG. 5 shows a detail of the matching circuit 70 and a structure of the table 68 of this embodiment. As shown, the table 68 has an upper plate portion 72 made of insulating material for supporting the implantation substrate 38 and a lower plate portion 74 made of conductive material and supporting the upper plate portion. The upper plate portion 72 includes at least one pair of chucking electrodes, a first electrode 76 and a second electrode 78, embedded therein. The first and second chucking electrodes 76 and 78 are connected to a DC power source 80 so that a certain DC voltage is applied between the chucking electrodes 76 and 78 to form an electrostatic force for holding the substrate 38 on the table 68.

The matching circuit 70 has a high frequency input terminal 82 which connects between the high frequency power source 52 and a capacitor 84. The terminal 82 is also connected through another capacitor 86, a coil 88, a capacitor 90, a low pass filter 92, and a monitoring circuit 94 with a potentiometer to another terminal 96 which is connected to the controller 60. Also, the opposite ends of the capacitor 90 are connected to a first output terminal 98 connected to the lower plate portion 74 of the table 68 and a second output terminal 100 connected to the annular monitoring electrode 66.

With the arrangement, a high frequency electric power is supplied from the power source 52 through the capacitor 86, the coil 88, the capacitor 90 and the output terminal 100 to the annular monitoring electrode 66. In this instance, the voltage of the annular monitoring electrode 66 is the same as that of the output terminal 100, so that a voltage which is in proportion to the DC voltage of the monitoring electrode 66 is obtained by the monitoring circuit 94. The obtained voltage, which also corresponds to the voltage of the table in the first embodiment, is then used at the controller 60 to control the implantation boron concentration.

Also in this matching circuit 70, the capacitor 90 separates the monitoring electrode 66 from the lower plate portion 74 of the table 68, which prevents a generation of a large negative voltage in the lower plate portion 74 which would cause a deterioration of the insulating, upper plate portion 72. The low pass filter 92 removes the high frequency power.

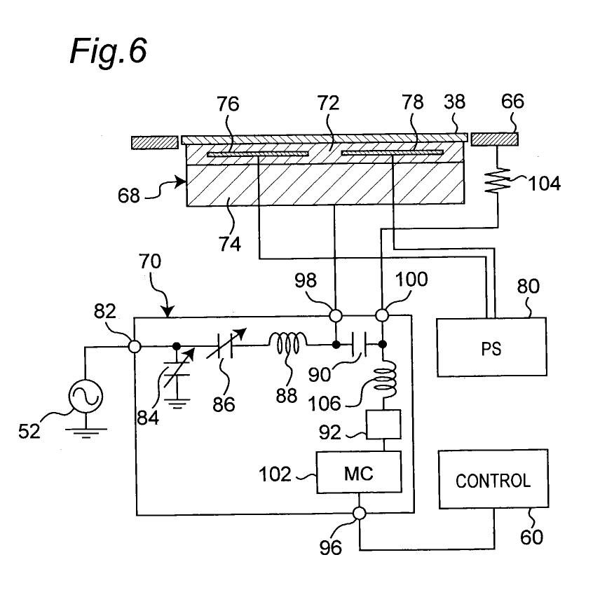

In the previous embodiment, the annular monitoring electrode is electrically floated in the circuit, so that no electric current flows in the circuit. Contrary to this, in order to flow an electric current in the circuit and thereby obtain a voltage using the detected current, modifications may be made to the circuit as shown in FIG. 6. Specifically, in this modification a monitoring circuit 102 in the matching circuit 54 has a first circuit part (not shown) for detecting an electric current flowing therethrough and a second circuit part (not shown) for calculating a voltage corresponding to the detected current. In addition, typically a resistor which is installed in the first circuit for detecting the electric current has a reduced resistance, which can result in an overheat and a resultant malfunctioning in the monitoring circuit. To prevent this, preferably a resistor 104 is connected in series to the annular monitoring electrode 66 to reduce the electric current flowing into the monitoring circuit. Also, as shown in FIG. 6 an additional coil 106 may be connected between the capacitor 90 and the monitoring circuit 102 to prevent a high frequency current from flowing into the monitoring circuit 102.

Tests were conducted by the use of the implantation device shown in FIG. 6. In the tests, the substrate was positioned on the table. The implantation gas mixture including Ar and B₂H₆ was supplied into the chamber. Amounts of argon and diborane were controlled to 10 sccm (standard cubic centimeters per minute) and 5 sccm, respectively. The pressure in the chamber was maintained at 0.04 Pa. Under the condition, the spiral coil and the table (the lower plate portion) were applied with high frequency powers from the power sources 50 and 52, respectively. As a result, it was confirmed that the boron was implanted in the surface of the substrate.

Also, in the tests the high frequency powers to the spiral coil and the table (the lower plate portion) were changed. Simultaneously, detected were the electric current flowing in the monitoring electrode and the implanted boron concentration in an interior of the substrate, spaced 1.0 nm away from the top surface of the substrate.

The result showed that the boron concentration increases substantially in proportion to the ion current density if the DC current flowing in the annular electrode is kept constant and, on the other hand, that the boron concentration increases substantially in proportion to the DC current in the annular electrode if the ion current density is kept constant. This means that the boron concentration is controlled in a very precise manner by controlling the high frequency power to the spiral coil to keep the ion current density constant and also controlling another high frequency power to the table to keep the current in the monitoring electrode constant.

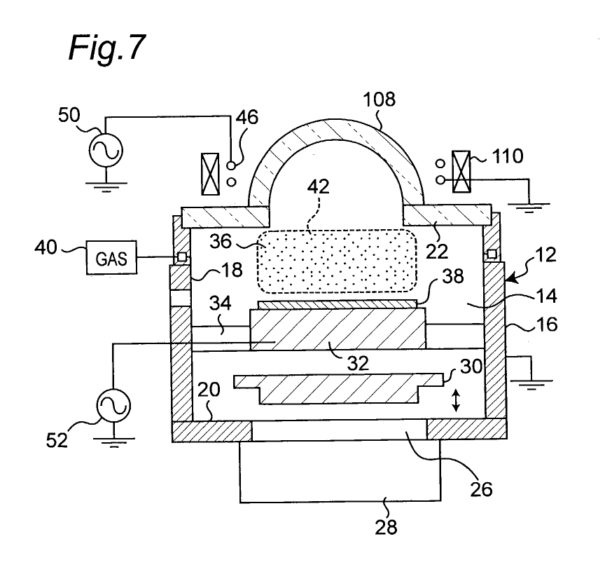

Although various embodiments have been described so far, the implantation device of the present invention may be modified and/or improved in various manners. For example, as shown in FIG. 7, a semidome top wall 108 may be used instead for the plate-like top wall in FIGS. 1 and 4. In this embodiment, a coil may be arranged in a non-spiral fashion. Also, a magnetic coil 110 for generating a magnetic field passing through the top wall toward the substrate may be provided, which allows to generate a helicon wave plasma or a magnetic neutral loop plasma, each having an elevated density than the inductively coupled plasma. Alternatively, a combination of a microwave emission antenna and the magnetic coil may be used. In this embodiment, an electron cyclotron resonance plasma is generated in the chamber, which has an elevated density than the inductively coupled plasma. In these modifications, a DC magnetic field or a low frequency magnetic field less than 1 kHz may be generated in the chamber by controlling the electric current flowing in the magnetic coil.

Also, although the semiconductor plate made of silicon is used for the substrate, it may be made of any material.

Further, although the boron is used for the implantation impurity, i.e., dopant, another impurity including arsenic, phosphorus, aluminum, and antimony may be implanted instead or additionally.

Further, although argon Ar is used for the dilution gas, it may be replaced with another gas made of nitrogen and helium, for example.

Furthermore, although the impurity is introduced in the gaseous form, i.e., B₂H₆, the impurity may be integrated in or on a certain substrate (impurity supply) and then is separated therefrom by sputtering, for example, into the chamber.

In addition, although the optical emission spectroscopy and the single probe method have been described in the previous embodiments for monitoring the condition of plasma in the chamber, another method can be used instead, including laser induced fluorescence method, infrared laser absorption spectroscopy, vacuum ultra violet absorption spectroscopy, laser scattering method, double probe method, triple probe method and quadrupole mass spectroscopy.

Also, although the voltage to be applied to the table is monitored in the previous embodiments, an electric current flowing therethrough may be monitored instead.

Further, although the voltage and current in the monitoring electrode are monitored in the previous embodiments, a high frequency current therein may be monitored instead.

### PARTS LIST

10: ion implantation device

12: container

14: chamber

16: first portion of container

18: side wall

20: bottom wall

22: second portion of container

24: top wall

26: opening

28: vacuum pump

30: valve member

32: table

34: support

36: space

38: substrate

40: plasma gas supply source

42: plasma

44: spiral coil

46: central end portion of coil

48: peripheral end portion of coil

50: first high frequency power source

52: second high frequency power source

54: matching circuit

56: voltage detector (second monitor)

58: light detector (first monitor)

60: controller

62: prove (single probe)

64: current density monitoring device

66: annular monitoring electrode

68: table

70: matching circuit

72: upper plate portion

74: lower plate portion

76: first electrode

78: second electrode

80: DC power source

82: terminal

84, 86: capacitor

88: coil

90: capacitor

92: low pass filter

94: monitoring circuit

96: terminal

98: output terminal

100: output terminal

102: monitoring circuit

104: resistor

106: coil

What is claimed is:

1. An apparatus for plasma implantation, comprising:
a vacuum container defining a vacuum chamber therein;
a table provided in the chamber for supporting a substrate to which an impurity is implanted;
a plasma generating element provided outside the chamber;
a first power source for applying a first high frequency electric power to the element to form a plasma in the chamber;
a second power source for applying a second high frequency electric power to the table;
a first detector for detecting a condition of the plasma;
a second detector for detecting a voltage or a current in the table; and
a controller for controlling at least one of the first and second high frequency electric power according to the condition of the plasma detected by the first detector and/or the voltage or the current detected by the second detector, thereby controlling an implantation concentration of the impurity to be implanted.

2. The apparatus of claim 1, wherein the first detector detects the condition using a method selected from an optical emission spectroscopy, a single probe method, a double probe method, a triple probe method, a laser induced fluorescence method, an infrared laser absorption spectroscopy, a vacuum ultra violet absorption spectroscopy, a laser scattering method and a quadrupole mass spectroscopy.

3. An apparatus for plasma implantation, comprising:
a vacuum container defining a vacuum chamber therein;
a table provided in the chamber for supporting a substrate to which an impurity is implanted;
a plasma generating element provided outside the chamber;
a first power source for applying a first high frequency electric power to the element to form a plasma in the chamber;
a second power source for applying a second high frequency electric power to the table;
an electrode provided adjacent the table and connected through a capacitor to the table;
a first detector for detecting a condition of the plasma;
a second detector for detecting a voltage or a current in the electrode; and
a controller for controlling at least one of the first and second high frequency electric power according to the condition of the plasma detected by the first detector and/or the voltage or the current detected by the second detector, thereby controlling an implantation concentration of the impurity to be implanted.

4. The apparatus of claim 3, wherein the first detector detects the condition using a method selected from an optical emission spectroscopy, a single probe method, a double probe method, a triple probe method, a laser induced fluorescence method, an infrared laser absorption spectroscopy, a vacuum ultra violet absorption spectroscopy, a laser scattering method and a quadrupole mass spectroscopy.

5. A method for impurity implantation into a substrate, comprising:
positioning a substrate on a table provided within a chamber;
generating a vacuum in the chamber;
supplying an impurity into the chamber;
applying a first high frequency electric power to a plasma generating element to thereby cause a plasma so that the impurity in the chamber is implanted in the substrate;
applying a second high frequency electric power to the table;
detecting a condition of the plasma in the chamber;
detecting a voltage or current in the table; and
controlling at least one of the first and second high frequency electric power according to the detected condition of the plasma and/or the detected voltage or current, thereby controlling an implantation concentration of the impurity to be implanted in the substrate.

6. The method of claim 5, wherein a frequency of the power from each of the first and second power sources is controlled in a range from 300 kHz to 3 GHz.

7. A device, having a member made from a substrate to which an impurity is implanted by the method of claim 5.

8. A method for impurity implantation into a substrate, comprising the steps of:
positioning a substrate on a table provided within a chamber;
generating a vacuum in the chamber;
supplying an implantation impurity into the chamber;
applying a first high frequency electric power to an element to thereby cause a plasma so that the impurity in the chamber is implanted in the substrate;
applying a second high frequency electric power to the table;
detecting a condition of the plasma in the chamber;
detecting a voltage or current in an electrode connected through a capacitor to the table; and
controlling at least one of the first and second high frequency electric power according to the detected condition of the plasma and/or the detected voltage or current, thereby controlling an implantation concentration of the impurity to be implanted.

9. The method of claim 8, wherein a frequency of the power from each of the first and second power sources is controlled in a range from 300 kHz to 3 GHz.

10. A device, having an element made from a substrate to which an impurity is implanted by the method of claim 8.
